(19) United States
(12) Patent Application Publication
(10) Pub. No.: US 2005/0075528 A1
(43) Pub. Date: Apr. 7, 2005

(54) Proess for desulfurization, denitrating and/or dearomatization of a hydrocarbon feedstock by adsorption on a spent solid adsorbent

(76) Inventors: Thorsten Burkhardt, Lyon (FR); Alexandre Nicolaos, Puteaux (FR); Fabrice Diehl, Rueil Malmaison (FR)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 10/793,751

(22) Filed: Mar. 8, 2004

(30) Foreign Application Priority Data
Mar. 7, 2003 (FR) 03/02.831

(51) Int. Cl. C10G 25/00; C07C 7/12

(52) U.S. Cl. 585/820; 585/826; 208/299

(57) ABSTRACT

A process for purification by adsorption of a hydrocarbon feedstock that uses a novel adsorption agent is described. This adsorption agent is a spent solid adsorbent that has been used in a first application of catalytic treatment of hydrocarbon feedstocks, or of adsorption, and whose adsorption or catalytic performance levels are degraded such that its use for the first application has become impossible. The use of this spent solid adsorbent for the purification of hydrocarbon feedstocks by adsorption does not require specific treatment in advance, in particular not heat treatment.

### STATE OF THE ART

The invention relates to a process for purification by adsorption, either desulfurization, denitrating and/or dearomatization, of a hydrocarbon feedstock by using a spent solid adsorbent that has been used in a first application of catalytic treatment or of adsorption and that makes it possible to obtain contents of sulfur, nitrogen and/or aromatic compounds that are compatible with the required specifications.

Hydrocarbon feedstock is defined as a hydrocarbon fraction such as a gas oil, a distillate, a gasoline or a kerosene, or more generally any petroleum fraction with a boiling point of between 50° C. and 550° C.

The standards that relate to the contents of sulfur-containing compounds, nitrogen-containing compounds and aromatic compounds of petroleum fractions are becoming more and more stringent. Thus, it is provided to lower the sulfur content that is allowed in the gas oil to 10 ppm by weight starting in 2009 in Europe and to 15 ppm by weight starting in 2006 in the United States.

The catalytic hydrotreatment is the standard tool for lowering the level of sulfur-containing compounds, nitrogen-containing compounds and aromatic compounds in the petroleum fractions.

The adsorption processes are considered more and more often in addition to an existing hydrotreatment/hydrodesulfurization unit to attain stringent specifications. They have the advantage of operating under much less stringent pressure and temperature conditions than the hydrotreatment. In addition, contrary to the hydrotreatment, these processes do not require hydrogen.

Patent WO 01/42397 claims a process for desulfurization of a petroleum fraction on activated carbon, whereby the sulfur-containing compounds have been oxidized in advance by an oxidizing agent. The regeneration of the activated carbon is carried out in several stages using a liquid desorbent, preferably toluene, and nitrogen.

One method consists in, for example, desulfurizing the middle distillates by adsorption of benzothiophene derivatives on adsorbents that are based on activated carbon, activated coke, supported CoMo (cobalt/molybdenum), zeolites, alumina and/or silica as described in the U.S. Pat. No. 5,454,933. In this case, the desorption solvent is toluene or xylene.

The sulfur-containing, nitrogen-containing and aromatic compounds are adsorbed on alumina, activated carbon, silica, silica gel, and zeolites by physisorption mechanisms that use low-energy electrostatic forces. The specific surface area of these adsorbents is generally on the order of 50 to 2000 m2/g, and their equivalent diameter is generally from 0.01 to 10 mm. Their micropore volume, determined with nitrogen, is generally encompassed between 0.01 and 0.8 cm3/g, and their mesopore volume, determined with mercury, is generally between 0.02 and 0.6 cm3/g.

Another method consists in adsorbing the benzothiophene-type sulfur-containing compounds, carbazole-type nitrogen-containing compounds, and/or aromatic compounds on a complexing agent as described in Patent Application WO 02/24836A1 and Patent FR2814172.

These methods have shown their advantage but the price of adsorbents used is from about 2 to 40 ∈/kg, which seriously reduces the attraction of these processes, given the large amounts of adsorbent that are necessary for an application of purification of a hydrocarbon feedstock, such as a gas oil, for example.

Patents RU208428 1 describe a method for producing adsorbents from spent hydrocracking or hydrogenation catalysts. This production, however, requires a complex treatment to convert a spent catalyst into an adsorbent, and in particular a heat treatment.

### SUMMARY DESCRIPTION OF THE INVENTION

This invention is a process for purification by adsorption of a hydrocarbon feedstock that uses a novel adsorption agent.

Novel adsorption agent that will be called a spent solid adsorbent in the text below is defined here as a solid adsorbent or a catalyst that has been used in a first catalytic application or a first adsorption application, and whose use in the first application has become impossible, either because of the reduction in performance levels (effluent that no longer meets the specifications of the original process) or because of a prohibitive economic cost due to, for example, a tightening of the operating conditions that are necessary to attain the specifications. Tightening is defined as a toughening of at least one operating condition.

In the case where the initial application is catalytic, it is possible to cite the catalysts for hydrotreatments, hydrocracking or hydrogenation of a petroleum fraction.

In the case where the initial application is an adsorption, it is possible to cite the following solids: the activated carbon that is activated chemically or physically, alumina, silica, or silica gel, and zeolites of any type.

The use of this agent for the purification of hydrocarbon feedstocks does not require specific treatment in advance, in particular not heat treatment.

The regeneration of this spent solid adsorbent is carried out by means of a suitable desorbent that is characterized by its desorption capacity and the possibility of separating said desorbent from the purified petroleum fraction, typically by distillation. Generally, the water vapor or a desorbent with a high content of aromatic compounds will be used. Such a desorbent can be, for example, toluene, xylenes and mixtures thereof.

Some of these spent solid adsorbents have adsorption and regeneration performance levels in purification of hydrocarbon feedstocks that are close to those of standard adsorbents. Therefore, these spent solid adsorbents have the great advantage of being much less expensive. Actually, the cost of such a spent solid adsorbent is most often very low, or even zero.

Spent solid adsorbent is defined here as a solid adsorbent or a catalyst that has been used in a first catalytic or non-catalytic application, whose use in the initial application has become impossible either because of the reduction in performance levels (the effluent no longer meets the specifications of the original process), or because of the prohibitive economic cost because of, for example, a tightening of the operating conditions necessary to attain the specifications.

In general, the spent solid adsorbent will be a solid adsorbent or a catalyst that has lost a significant portion of its adsorption capacity or its initial activity because of a contamination during its first application. This contamination can be demonstrated in different ways. By way of example, it can be caused by blocking the access to active sites by clogging pores or by the deposition of various products initially contained in the feedstock that are deposited during this first application, such as, for example, coke and metals.

As a general criterion of the loss of adsorption or activity capacity, the concept of relative reduction of the specific surface area of the spent solid adsorbent will be used.

According to the initial use of the spent solid adsorbent, this concept optionally can be completed by more specific criteria, such as, for example, the coke content that is deposited on the spent solid adsorbent, or the content of certain metals.

Therefore, as a spent solid adsorbent, any solid adsorbent will be considered that has operated in a first catalytic or adsorption application and that has lost, following this first application, between 5% and 80% of its initial specific surface area and preferably between 10% and 60% of this initial specific surface area.

A particular spent solid adsorbent in this context is preferably a spent catalyst that is obtained from one of the following refining processes: hydrotreatment, hydrodesulfurization, hydrodenitrating, hydrodemetalization, hydrocracking or hydrogenation of a petroleum fraction, whereby the performance levels of this catalyst are degraded such that its use as a catalyst has become economically disadvantageous, either by the necessity of increasing the operating temperature in a significant way or because of a significant lowering of the selectivity relative to the new catalyst.

It has thus been found that some of these spent solid adsorbents are entirely effective as an adsorbent for the purification of hydrocarbon feedstocks and that their performance levels in adsorption and regeneration are close to those of standard adsorbents, such as alumina, activated carbon or zeolites.

It is possible, without this list being limiting, to cite as examples of spent solid adsorbent the following solids: catalysts for hydrotreatment of gas oils, catalysts for hydrotreatment of gasolines, catalysts for hydrocracking, activated carbon for the purification of air or gaseous effluents, solid for the purification of water.

Among the hydrodesulfurization catalysts that can be used, it thus is possible to use catalysts that comprise metals of group VIII and optionally metals of group VIB dispersed on a substrate. Such catalysts comprise, for example, nickel and/or cobalt and optionally molybdenum and/or tungsten, whereby these metals are dispersed on a substrate, for example an alumina or a silica-alumina.

The advantage of this invention resides in the cost of such a spent solid adsorbent whose market value is generally very low, or even negative in terms of where the user often winds up having to pay the recycler for this type of product.

Actually, the cost of recovering possible potentially advantageous components, such as metals, for example, is often close to the market price of these components, which imposes very low repurchase prices for these spent solid adsorbents.

After its second application, this spent solid adsorbent, object of this invention, optionally will require a suitable treatment or storage like any standard adsorbent.

The invention therefore consists of a process for purification by adsorption of a hydrocarbon feedstock in liquid or gaseous phase, constituting a second application, characterized by the use of a spent solid adsorbent that is a catalyst that has been used in a first catalytic application, or an adsorbent that has been used in a first adsorption application.

The spent solid adsorbent will generally have lost between 5 and 80% of its initial specific surface area, and preferably between 10 and 60% of this initial specific surface area, following its first application.

In some cases, the spent solid adsorbent can be a spent catalyst that is obtained preferably from one of the following refining processes: hydrotreatment, hydrodesulfurization, hydrodenitrating, hydrodemetalization, hydrocracking or hydrogenation of a petroleum fraction.

In other cases, the spent solid adsorbent can preferably be activated carbon that is activated chemically or physically, alumina, silica, or silica gel, or zeolites of any type.

The process for purification by adsorption according to the invention generally makes use of at least two adsorption columns that work by adsorption or by regeneration according to the so-called alternation technique between the adsorber and the regenerator (“swing”-type technique according to English terminology) that is well known to one skilled in the art.

The regeneration can itself be carried out in two stages:
A first stage during which a portion of the feedstock in the portion that is desulfurized, denitrated and/or dearomatized and that is contained in the pore volume of the adsorbent is washed by means of a desorbent, and an effluent is recovered from the first regeneration partly desulfurized, denitrated and/or dearomatized.
A second stage during which the desorbent is circulated in the adsorption column, and an effluent is recovered from the second regeneration that contains the sulfur-containing, nitrogen-containing and/or aromatic compounds that have been desorbed.

The desorbent that is used for the regeneration of the solid adsorbent will generally be selected from among the following compounds: toluene, xylenes, petroleum fractions with a high content of aromatic compounds, water vapor or any mixture of said compounds.

In some cases where, for example, desorbent will not be used under satisfactory economic conditions, it may be possible not to regenerate the spent solid adsorbent and simply to eliminate it.

The hydrocarbon feedstock that is to be treated will often be a gasoline, a gas oil, a kerosene, an atmospheric distillation residue, or more generally any petroleum fraction that contains sulfur-containing compounds, nitrogen-containing compounds and/or aromatic compounds.

### DETAILED DESCRIPTION OF THE INVENTION

In a more detailed way, the invention relates to a process for purification by adsorption, whereby the term purification by adsorption designates the desulfurization, the denitrating and/or the dearomatization of a hydrocarbon feedstock, advantageously with boiling points of between 50° C. and 550° C., containing sulfur-containing compounds, nitrogen-containing compounds and/or polyaromatic compounds.

These feedstocks generally will be atmospheric distillation gasolines, FCC (fluidized-bed catalytic cracking) gasolines, gas oils of all origins, and atmospheric distillation residues.

The process comprises an adsorption stage of the feedstock in at least one adsorption column that contains the spent solid adsorbent. An adsorption effluent that is at least partly desulfurized, denitrated and/or dearomatized is recovered, and after a certain time of operation, said column is regenerated by means of a suitable desorbent.

The adsorption and the regeneration can be carried out in liquid phase or in gaseous phase.

The effluents of the adsorption and the regeneration contain the purified hydrocarbon feedstock, the desorbent and sulfur-containing, nitrogen-containing and/or aromatic compounds. These components are then separated in at least one piece of separating equipment, which in some cases can be a decanter or a distilling column or any type of separation technology.

This invention is in no way linked to the separation technology that is used for separating the purified hydrocarbon feedstock from effluents obtained from the adsorber during adsorption and regeneration phases.

The preferred variant of the process, using an adsorption and a regeneration in liquid phase, calls for the regeneration of the adsorption column in two stages:
A first stage during which a portion of the feedstock that is partly desulfurized, denitrated and/or dearomatized and that is contained in the pore volume of the adsorbent is washed by means of a desorbent, and an effluent is recovered from the first regeneration partly desulfurized, denitrated and/or dearomatized.
A second stage during which the desorbent is circulated in the adsorption column, and an effluent is recovered from the second regeneration that contains sulfur-containing, nitrogen-containing and aromatic compounds that have been desorbed.

When the second regeneration phase has ended, the adsorption column passes into an adsorption phase. It is then possible to introduce the feedstock directly into the adsorption column. The purified hydrocarbon feedstock is then separated from the desorbent that is contained in the pore volume, generally by a downstream distillation column.

The regeneration in two stages can be carried out either continuously or intermittently.

Typically, the adsorption and the regeneration in liquid phase are carried out under similar pressure and temperature conditions, namely an absolute pressure, from 0.001 MPa to 3.0 MPa, preferably between 0.1 and 2.0 MPa, and a temperature that is less than 400° C., for example from 20° C. to 350° C., and preferably between 40° C. to 250° C.

However, different temperature and pressure levels between the adsorption phase and the regeneration phase can be considered so as to facilitate respectively the adsorption and the desorption of the sulfur-containing, nitrogen-containing and/or polyaromatic compounds.

Typically, the adsorption and the regeneration can be carried out at surface velocities
from 0.1 mm/s to 30 mm/s, preferably from 1 mm/s to 10 nm/s, if it is an adsorption or a regeneration in liquid phase.
from 1 cm/s to 120 cm/s, preferably from 3 cm/s to 50 cm/s, if it is an adsorption or a regeneration in gaseous phase.

The surface velocity is defined as the ratio of the volumetric flow rate of the feedstock to the conditions of the adsorber in the empty section of the column.

The volumetric flow rate during an adsorption or a regeneration in liquid phase is generally encompassed between 0.1 h⁻¹ and 15 h^{-1,} and preferably between 0.5 h⁻¹ and 2 h⁻¹, and it is generally encompassed between 1 h⁻¹ and 750 h⁻¹, and preferably between 5 h⁻¹ and 200 h⁻¹ during an adsorption or a regeneration in gaseous phase. The volumetric flow rate is defined as the ratio of volumetric flow rate of the feedstock under standard conditions to the volume of the adsorbent.

Advantageously, the desorbent, on the one hand, and the hydrocarbon feedstock that is partially purified as well as the impurities that are desorbed, on the other hand, contained in the adsorption effluent are generally separated by distillation into the first regeneration effluent and into the second regeneration effluent. The desorbent can be recycled in the column where the regeneration is carried out.

The boiling point of the regeneration desorbent preferably makes it possible to separate it from the hydrocarbon feedstock by distillation.

Advantageously, the process according to this invention can be located upstream or downstream from a hydrotreatment unit.

Advantageously, the second regeneration effluent that contains desorbed impurities can be sent upstream from a hydrotreatment unit.

According to another characteristic, if the hydrocarbon feedstock can be divided into a fraction that is high in sulfur-containing, nitrogen-containing and/or polyaromatic compounds and into a fraction that is low in sulfur-containing, nitrogen-containing and/or polyaromatic compounds, for example by distillation, only the fraction that is high in sulfur-containing, nitrogen-containing and/or polyaromatic compounds is introduced as a feedstock into the adsorption column.

Advantageously, it is possible to operate the adsorption stage of the feedstock in at least one adsorption column while the regeneration stage is operated in at least one other column, whereby the two columns work alternately in adsorption phase and in regeneration phase.

According to another characteristic, the spent solid adsorbent is not regenerated after its use in the process of this invention, but it is eliminated.

According to another characteristic, the regeneration can be carried out under operating conditions that are different from those of the adsorption, and in particular at a lower pressure and a higher temperature for facilitating the regeneration.

According to another characteristic, the hydrocarbon feedstock that is contained in the pore volume during the alternations between the adsorption and the regeneration can be washed with a hot gas, such as, for example, nitrogen, hydrogen or water vapor. In this case, the separations can be carried out by decanting.

The adsorbers are typically fixed beds according to the alternation technique that is described below, but the invention can also be carried out by any other type of process such as the moving bed or the fluidized bed. This alternation technique between at least two adsorbers, one passing from the adsorption phase to the regeneration phase, the other passing simultaneously from the regeneration phase to the adsorption phase, is also called “swing” according to English terminology.

Because an effort is made to use the spent solid adsorbent for its very low cost, or even zero cost, it is preferable not to increase this cost further by adding on the cost of treatments whose object is, for example, to modify the grain size, although such a treatment upstream from the second application remains within the scope of this invention.

In this case, the selection of the technology of the second application in a fixed bed, moving bed or fluidized bed will in large part be dictated by the grain size of the spent solid adsorbent such as that resulting from its first application.

According to an advantageous characteristic of the process, it is possible to mix the adsorption effluent and the first regeneration effluent, to distill the mixture to eliminate the desorbent from it, and to recover a hydrocarbon effluent to the required specifications of sulfur, nitrogen and/or aromatic compounds.

According to another characteristic, the second regeneration effluent can be distilled, and a residue that is high in sulfur-containing, nitrogen-containing and/or aromatic compounds and a distillate that contains the desorbent that is recycled in the column where the regeneration is carried out are recovered. Said residue can be downgraded into a product with a market value that is lower than that of the purified hydrocarbon feedstock. By way of example, if the hydrocarbon feedstock to be purified is a gas oil, the residue can be upgraded as a domestic fuel.

Advantageously, the fraction that is low in sulfur-containing, nitrogen-containing and/or polyaromatic compounds of the hydrocarbon feedstock, described above, can be mixed with the hydrocarbon feedstock that is obtained from the adsorption effluent and/or the first regeneration effluent after distillation, and/or the mixing of the two preceding effluents to obtain a fuel with the required specifications of sulfur, nitrogen and/or aromatic compounds.

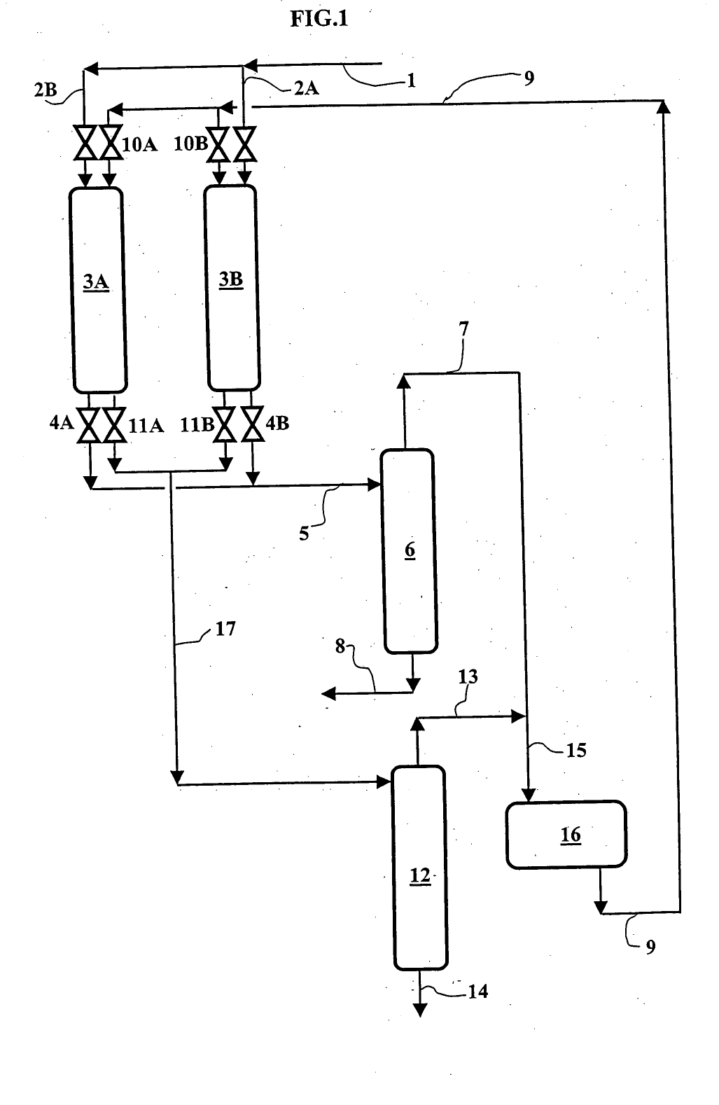

The use of the spent solid adsorbent in the process according to the invention will be better understood based on FIG. 1 that diagrammatically illustrates the process of adsorption and regeneration of the adsorbent in the case of the final desulfurization of a gas oil.

Gas oil is defined as a petroleum fraction whose boiling point is between about 150° C. and 450° C. This case does not at all limit the scope of the invention to other petroleum fractions and to other compounds to be removed, in particular nitrogen-containing, neutral and basic compounds, and polyaromatic compounds.

According to FIG. 1, the gas oil that is to be desulfurized (1) is sent to the adsorption/desorption treatment that comprises at least two adsorbers (3a) and (3b), filled with spent solid adsorbent, whereby at least one of the adsorbers is in adsorption phase, and the others are in regeneration phase. Below, for the sake of simplicity, the invention is described for a case that comprises two adsorbers. Gas oil that is to be purified (1) is therefore sent by means of a system (2a) of suitable pipes and valves to adsorber (3a), in which the compounds that are to be eliminated, primarily the sulfur-containing compounds in this example of application, are selectively adsorbed on the spent solid adsorbent.

Effluent (4a) that is obtained from adsorber (3a) essentially contains purified gas oil, but also a non-negligible fraction of desorbent. Actually, each time that an adsorber is put back into adsorption mode after having been regenerated, it is still filled with the desorbent that was used in regenerating it.

Effluent (4a) is then directed through a line (5) to a distillation column (6) that separates purified gas oil (8), on the one hand, and desorbent (7), on the other hand.

As the adsorbent that is contained in adsorber (3a) loads up on sulfur-containing compounds, the sulfur content in effluent (4a) increases. When this content reaches a sulfur limit threshold (followed by regular analyses of the sulfur content of effluent (4a)), adsorber (3a) passes, by means of pipes and valves (2a) and (2b) from the adsorption phase to the regeneration phase, while adsorber (3b) passes from the desorption phase to the adsorption phase.

The adsorber that passes from the adsorption phase to the regeneration phase also contains gas oil in the pore volume, a volume that comprises the interstices and the pores of the adsorption agent. The sulfur content of this gas oil is between that of gas oil (1) and that of purified gas oil (4a).

The regeneration of the adsorption agent consists in entraining the sulfur-containing, nitrogen-containing and aromatic compounds by elution by means of the desorbent. The regeneration according to the preferred embodiment is carried out in two phases.

Phase 1 of the regeneration: desorbent (9), which is obtained from an outside source or from the recycled desorbent of the process, is injected into adsorber (3b), for example, by means of suitable pipes and valves (10b). During this first phase of the regeneration, the effluent of adsorber (3b) contains a significant portion of the gas oil that is obtained from the pore volume and a small amount of desorbent. The contents of sulfur, nitrogen and aromatic compounds of this portion of the gas oil are found among those of gas oil before the treatment by adsorption according to the invention and those of gas oil that is obtained from the adsorption phase. This phase of the regeneration comes to an end when the sulfur content in effluent (4b) begins to increase significantly because of the appearance of desorbed sulfur-containing compounds.

This is generally the case after the injection of a volume of desorbent that corresponds to 30% to 700% of the pore volume of the adsorption agent. Because of its low sulfur content, this portion of effluent (4b) can be mixed with the effluent of adsorption zone (4a), the whole mixture being injected into distillation column (6). Then, a top effluent (7) that contains the desorbent and a bottom effluent (8) that contains the desulfurized gas oil are recovered.

The sulfur limit thresholds for which the adsorption phase and the first regeneration phase are stopped are selected such that the content of sulfur, nitrogen and/or polyaromatic compounds of the gas oil obtained from the mixing of lines (4a) and (4b) is less than or equal to the required specification.

Phase II of the regeneration begins as soon as the sulfur content in effluent (4b) of the first phase of regeneration of adsorber (3b) increases significantly following the appearance of the desorbed sulfur-containing compounds.

The effluent of adsorber (3b) then consists of the remainder of the gas oil of the pore volume (the portion that has not left the adsorber during the first phase of regeneration) and desorbent that contains the desorbed sulfur-containing compounds.

This effluent of the second phase of regeneration (11b) is directed, by means of suitable pipes and valves, to a distillation column (12) that separates desorbent (13), on the one hand, and a sulfur-containing gas oil (14), on the other hand.

Taking into account their range of boiling points, which is identical to that of gas oil, the sulfur-containing compounds recombine and are released together with gas oil (14) from column (12). Desorbent flows (7) and (13) at the outlet of columns (6) and (12) advantageously can be regrouped in a tank (16) and recycled in the process to adsorbers (3a) or (3b). The flow of purified gas oil (8) constitutes the gas oil that complies with the specifications.

Flow (14) that is obtained at the bottom of column (12) that contains virtually all of the desorbed sulfur-containing compounds can in some cases be recycled in a unit such as a unit for hydrodesulfurization of gas oils or a hydrocracking when one of these units exists in the vicinity. In other cases, it undergoes a downgrading, i.e., it is marketed at a lower market value than the purified gas oil, but its sulfur content remains low enough so that this effluent can keep a high market value.

Regarding the second phase of regeneration, a volume of desorbent that corresponds to 30 to 5000% of the pore volume of the adsorption agent may be necessary to desorb the adsorbed compounds and preferably 300 to 1000% of the pore volume.

The advantages of this preferred embodiment reside in the fact that thanks to the two-phase regeneration, it is possible, on the one hand, to avoid the downgrading of the gas oil fraction that is contained in effluent (4b), and, on the other hand, the quality of the gas oil fraction that corresponds to flow (14) is adequate to constitute a product that can be upgraded. The processing/storage problems of sulfur-containing, nitrogen-containing and/or aromatic compounds that are extracted are thus totally eliminated.

It has been shown by experiment that it thus is possible to recover between 30% and 100% of the gas oil, at least partially desulfurized, denitrated and/or dearomatized and contained in the pore volume, during the first regeneration phase. Only 0% to 70% of the remainder of the gas oil contained in the pore volume of the adsorbent is either to be recycled before hydrotreatment or hydrocracking or to be downgraded, taking into account the sulfur content. Advantageously, if the downgrading proves necessary, this gas oil can be downgraded into a home heating fuel. Relative to a regeneration in a single phase that leads to the recycling or to the downgrading of all of the gas oil that is contained in the pore volume of the adsorbent, this represents a substantial economic advantage.

### EXAMPLE

Tests of adsorption and regeneration are carried out by using two spent solid adsorbents, a spent catalyst for hydrotreatment of gas oils and a spent catalyst for hydrotreatment of ex-FCC gasolines (fluidized-bed catalytic cracking gasoline or fluid catalytic crackings according to the English terminology), as well as a standard adsorbent, activated carbon that is activated chemically.

In their current states, the spent catalyst for hydrotreatment of gas oils requires an increase in temperature of 19° C., and the catalyst for hydrotreatment of gasolines requires an increase in temperature of 31° C. relative to the new catalysts to reach the desulfurization levels of the new catalysts. Such an increase in the temperature would limit the economic advantage of the hydrotreatment process, on the one hand, and would promote the formation of coke on the catalyst, leading to an accelerated loss of activity, on the other hand.

The following table summarizes the properties of the two hydrotreatment catalysts that were tested:

| | Spent Catalyst for | Spent Catalyst for |
|---|---|---|
| | Hydrodesulfurization of | Hydrodesulfurization |
| | Gas Oils | of Gasolines |
| Specific Surface Area | 183 m2/g | 147 m2/g |
| Initial Specific Surface | 205 m2/g | 200 m2/g |
| Area | | |
| Form | Trilobar Extrudates | Cylindrical Extrudates |
| Diameter | 1.2 mm | 1.2 mm |
| Loading Density | 0.78 kg/l | 0.78 kg/l |
| Pore Volume | 0.44 cm3/g | 0.50 cm3/g |
| Substrate Material | Al2O3 | Al2O3 |
| Contents of Metals | | |
| Nickel (NiO) | 3.3% by Weight | — |
| Molybdenum (MoO3) | 16.5% by Weight | 14.0% by Weight |
| Cobalt (CoO) | — | 3.0% by Weight |
| Coke | 7.6% by Weight | 5.2% by Weight |
| Poisons | | |
| Iron (Fe) | 560 ppm by weight | 400 ppm by weight |
| Gallium (Ga) | <100 ppm by weight | 100 ppm by weight |
| Nickel (Ni) | | 280 ppm by weight |
| Selenium (Se) | — | 100 ppm by weight |
| Silica (Si) | 400 ppm by weight | 8000 ppm by weight |
| Titanium (Ti) | — | 350 ppm by weight |
| Chlorine (Cl) | 250 ppm by weight | — |
| Calcium (Ca) | <100 ppm by weight | — |
| Copper (Cu) | <100 ppm by weight | — |

Activated carbon that is activated chemically with a specific surface area of 1442 m2/g and with a specific loading mass of 400 kg/m3 was used.

Adsorption and regeneration tests are aimed at purifying, i.e., desulfurizing, denitrating and dearomatizing, a hydrocarbon feedstock. Involved is a model gas oil that consists of:
70.6% by weight of heptane,
50 ppm by weight of sulfur in the form of dibenzothiophene (DBT),
40 ppm by weight of nitrogen in the form of indole,
22.4% by weight of monoaromatic compounds in the form of PDEB (para-diethyl benzene),
6.4% by weight of diaromatic compounds in the form of naphthalene,
0.40/o by weight of triaromatic compounds in the form of phenanthrene.

The regeneration solvent is toluene.

The adsorption was carried out at ambient temperature and at atmospheric pressure in liquid phase. The regeneration was carried out by toluene at 200° C. and at atmospheric pressure in the gaseous phase.

The adsorption capacities relative to the various components and the amount of desorbent that is necessary for regenerating the adsorbent are determined by the piercing and depiercing curves. For this purpose, a column with an inside diameter of 1 cm is loaded with 22 ml of adsorbent. The flow rates of hydrocarbon feedstock and desorbent are 5 ml/min.

In a first step, the hydrocarbon feedstock is injected, and the piercing curves of the different compounds are determined (i.e., the curves for evaluating the content of these compounds as output of the adsorber).

In a second step, when the adsorbent is saturated with sulfur, nitrogen, monoaromatic compounds, diaromatic compounds and triaromatic compounds, the desorbent is injected to regenerate the solid. This regeneration stage comes to an end when the sulfur content in the effluent is less than the detectability limit of 1 ppm by weight of sulfur.

The following table summarizes the performance levels of adsorption and regeneration that are determined. These are:
the sulfur adsorption capacity that is expressed by mass of sulfur adsorbed per solid mass
the nitrogen adsorption capacity that is expressed by mass of nitrogen adsorbed per solid mass
the monoaromatic compound adsorption capacity that is expressed by mass of monoaromatic compound adsorbed per solid mass
the diaromatic compound adsorption capacity that is expressed by mass of diaromatic compound adsorbed per solid mass

the amount of injected toluene that is necessary to obtain an adsorber effluent whose sulfur content is less than the detectability limit.

| | | |
|---|---|---|
| Spent Catalyst for | Spent Catalyst for | |
| Hydrodesulfurization | Hydrodesulfurization | Activated Carbon |
| of Gas Oils | of Gasolines | (ChemicallyActivated) |

Adsorption: Adsorption Capacities

| | | | |
|---|---|---|---|
| Adsorption | 0.091 | 0.021 | 0.463 |
| Capacity of | | | |
| Sulfur | | | |
| q_{sulfur} of | | | |
| mg_{nitrogen}/g_{adsorbent} | | | |
| Adsorption | 0.49 | 0.63 | 1.02 |
| Capacity of | | | |
| Nitrogen | | | |
| q_{nitrogen} of | | | |
| mg_{nitrogen}/g_{adsorbent} | | | |
| Adsorption | 16.7 | 16.6 | 324 |
| Capacity of | | | |
| Monoaromatic | | | |
| Compounds | | | |
| q_{monoaromaticcompounds} | | | |
| of | | | |
| mg_{monoaromaticcompounds}/ | | | |
| g_{adsorbent} | | | |
| Adsorption | 13.0 | 12.7 | 107 |
| Capacity of | | | |
| Diaromatic | | | |
| Compounds | | | |
| q_{diaromaticcompounds} | | | |
| of mg_{diaromaticcompounds}/ | | | |
| g_{adsorbent} | | | |

Regeneration

| | | | |
|---|---|---|---|
| Amount of | 2.67 | 2.40 | 2.69 |
| Toluene to | | | |
| Desorb and | | | |
| Release Sulfur | | | |
| (Vₜₒₗᵤₑₙₑ/V_{adsorbent}) | | | |

The adsorption capacities of the two spent hydrotreatment catalysts for the various components that are contained in the gas oil are quite obviously less than those of activated carbon, in particular concerning the desulfurization due primarily to the difference in specific surface area.

In contrast, the amount of desorbent that is necessary to regenerate the adsorbent is essentially the same for the three solids being considered.

By way of example, the selective desulfurization of a gas oil that is released from a hydrodesulfurization unit, of 50 to 10 ppm by weight of sulfur by adsorption, is considered. The composition of this gas oil is equal to that of the model feedstock, and the adsorption is carried out at atmospheric pressure and at ambient temperature. A volumetric flow rate of 1.2 h⁻¹ is considered for the adsorption. A system of adsorbers working in alternating fashion, i.e., alternating between the adsorption and the regeneration, is used.

The reduced sulfur adsorption capacity of the spent catalysts relative to the activated carbon is reflected by a faster piercing of the sulfur-containing compounds at the outlet of the adsorbers. This is offset by an increase of the alternation between the adsorption and the regeneration, leading to an increase in the amount of gas oil to be downgraded.

Actually, each time that an adsorber is regenerated, a portion of the gas oil that is contained in the pore volume will be downgraded.

Under these conditions, a volume of adsorbent that corresponds to 1/1.2 m³ per m³/h of gas oil that is to be treated, corresponding to the volumetric flow rate of 1.2 h⁻¹ per adsorber, is necessary. The total amount of adsorbent is multiplied by a factor of 2 because of the alternating operation, which assumes two adsorbers.

The use of the spent solid adsorbent whose market value is generally very low, as explained above, leads to a considerable savings relative to the use of a standard adsorbent, such as the activated carbon in our example.

If a gas oil flow rate of 166 m3/h, corresponding to a mean hydrotreatment unit with a capacity of 25,000 barrels/day, is considered, an adsorbent amount of 276 m³ is necessary.

If a cost of activated carbon of 10 ∈/kg is considered, the adsorbent position's cost comes to 1104 k∈, and it is necessary to make this investment at a frequency that corresponds to the service life of the adsorbent. If a typical adsorbent service life of 4 years is considered, the savings realized on this position, due to the use of the spent solid adsorbent, will therefore be 276 k∈ per year, which is quite significant.

1. Process for purification by adsorption of a hydrocarbon feedstock in liquid or gaseous phase, constituting a second application, characterized by the use of a spent solid adsorbent that is a spent catalyst that is obtained from one of the following refining processes: hydrotreatment, hydrodesulfurization, hydrodenitrating, hydrodemetalization, hydrocracking or hydrogenation of a petroleum fraction.

2. Process for purification by adsorption according to claim 1, in which following its first application, the spent solid adsorbent lost between 5 and 80% of its initial specific surface area, and preferably between 10 and 60% of this initial specific surface area.

3. Process according to claim 1, in which the spent catalyst is a spent catalyst for hydrodesulfurization of gas oils or gasolines.

4. Process for purification by adsorption according to claim 1, in which according to the alternation technique, an adsorption stage is carried out in at least one adsorption column, and a regeneration stage is carried out in at least one other adsorption column by means of a suitable desorbent, whereby said regeneration is carried out in two stages:
A first stage during which a portion of the feedstock in the portion that is desulfurized, denitrated and/or dearomatized and that is contained in the pore volume of the adsorbent is washed by means of a desorbent, and an effluent is recovered from the first regeneration partly desulfurized, denitrated and/or dearomatized.
A second stage during which the desorbent is circulated in the adsorption column, and an effluent is recovered from the second regeneration that contains the sulfur-containing, nitrogen-containing and/or aromatic compounds that have been desorbed.

5. Process for purification by adsorption according to one claim 1, in which the spent solid adsorbent is not regenerated.

6. Process for purification by adsorption according to claim 1, in which the desorbent that is used for the regeneration of the spent solid adsorbent is selected from among the following compounds: toluene, xylenes, petroleum fractions with a high content of aromatic compounds, water vapor or any mixture of said compounds.

7. Process for purification by adsorption according to claim 1, in which the hydrocarbon feedstock that is to be treated is a gasoline, a gas oil, a kerosene, or an atmospheric distillation residue.
